(19) United States
(12) Patent Application Publication
(10) Pub. No.: US 2023/0248204 A1
(43) Pub. Date: Aug. 10, 2023

(54) COMPANION ANIMAL MANAGEMENT APPARATUS

(71) Applicant: Hyunjin JUNG, Seoul (KR)

(72) Inventor: Hyunjin JUNG, Seoul (KR)

(21) Appl. No.: 18/135,871

(22) Filed: Apr. 18, 2023

Related U.S. Application Data
(63) Continuation of application No. PCT/KR2021/013262, filed on Sep. 28, 2021.

(30) Foreign Application Priority Data
Oct. 19, 2020 (KR) 10-2020-0135362

(51) Int. Cl. A47L 11/40; A01K 1/01

(52) U.S. Cl. CPC A47L 11/4044; A01K 1/011; A47L 11/4066; A47L 11/4069; A47L 11/4088; A47L 11/4011; A47L 2201/04; A47L 2201/06

(57) ABSTRACT

A companion animal management apparatus is provided. The companion animal management apparatus includes a main body, a driving module configured to move the main body, and a cleaning module installed in an interior space of the main body and including a part which protrudes out to the front of the main body and retreats back into the main body to process feces inside the main body.

### CROSS REFERENCE TO RELATED APPLICATIONS

This is a continuation application of International Patent Application No. PCT/KR2021/013262 filed on Sep. 28, 2021, which claims priority to Korean Patent Application No. 10-2020-0135362 filed on Oct. 19, 2020, contents of all of which are incorporated herein by reference.

### TECHNICAL FIELD

The present disclosure relates to a companion animal management apparatus.

### BACKGROUND ART

With the highly developed industry and abundant life environment, the number of companion animals living in households has gradually increased. When keeping a pet, providing a fixed amount of food in a timely manner is required to resolve nutrient deficiency and overeating issues. However, when all family members are not home or away from home for a long time due to vacation, etc., or when a working couple or a single household keeps a pet, the pet stays alone in the house and may not be properly cared.

To resolve this problem, technologies for managing companion animals and distributing food by using a robot have been developed from various angles, and Korean Patent Registration No. 10-1721386 entitled “automatic feeding movable apparatus, care robot for companion animal, care system for companion animal having the same, and control method thereof” registered on Mar. 23, 2017 discloses a companion animal care robot to overcome the aforementioned problem. However, as existing companion animal management robots simply supply food to the companion animals or are connected to an administrator terminal to manage the companion animals, management services such as accurately identifying health condition of the companion animals and providing proper food and exercise considering the current condition of the companion animals could not be provided.

When a companion animal is not trained to defecate on an absorbent pad for pet, or defecates at various locations in the house, it is essential to find and clean the feces. As there is a need for cleaning devices designed specifically for companion animals, research thereon is needed.

### SUMMARY

### Technical Problem

The present disclosure provides a companion animal management apparatus which increases activity of companion animals, monitors condition of companion animals, and processes feces of companion animals.

### Technical Solution

An aspect of the present disclosure provides a companion animal management apparatus including a main body, a driving module configured to move the main body, and a cleaning module installed in an interior space of the main body and including a part which protrudes out to the front of the main body and retreats back into the main body to process feces inside the main body.

### Advantageous Effects

A companion animal management system and a companion animal management apparatus according to the present disclosure may monitor condition of a companion animal and deliver the same to a user terminal such that a user may check and manage the condition of the companion animal in real time. The companion animal management apparatus may obtain a current image of the companion animal such that a user may check on the companion animal in real time.

According to the companion animal management system and the companion animal management apparatus of the present disclosure, the companion animal management apparatus may obtain data about activity of the companion animal such that condition of the companion animal is closely managed. By measuring data about feces, movement distance, etc. of the companion animal, condition of the companion animal may be assumed, and the amount of food or treat to be fed may be adjusted.

The companion animal management system and the companion animal management apparatus according to the present disclosure may clean up feces of the companion animal. A cleaning module may accurately collect and remove the feces. In addition, a spray module may spray liquid chemical to remove odor of feces and quickly harden the feces.

The companion animal management system and the companion animal management apparatus according to the present disclosure may set a cleaning route based on data about positions where the companion animal has previously defecated. By first cleaning the position where the companion animal has defecated, dispersion of contamination may be prevented. However, the scope of the present disclosure is not limited to the above mentioned effects.

### DESCRIPTION OF DRAWINGS

FIG. 1 is a diagram of a companion animal management system according to an embodiment of the present disclosure.

FIG. 2 is a diagram of a companion animal management apparatus according to an embodiment of the present disclosure.

FIG. 3 is a block diagram illustrating some components of the companion animal management apparatus of FIG. 2.

FIGS. 4A to 4E are each a cross-sectional diagram illustrating operation of the companion animal management apparatus of FIG. 2.

FIGS. 5A to 5C are cross-sectional diagrams illustrating operation of the cleaning module of FIG. 3.

FIG. 6 is a diagram illustrating a method of controlling operation of the companion animal management apparatus of FIG. 2.

### DETAILED DESCRIPTION

An aspect of the present disclosure provides a companion animal management apparatus including a main body, a driving module configured to move the main body, and a cleaning module installed in an interior space of the main body and including a part which protrudes out to the front of the main body and retreats back into the main body to process feces inside the main body.

The cleaning module may include a first moving unit fixed to the main body and configured to linearly move a first moving portion, a second moving unit connected to the first moving portion, linearly moving along with the first moving unit, and configured to linearly move a second moving portion, a plate connected to the second moving portion, and a cover film including both ends fixed to a connection block arranged outside the second moving unit and having an area covering an upper surface of the plate, the area changing according to linear movement of the second moving portion.

The companion animal management apparatus may further include a spray module arranged on the front of the main body and configured to spray liquid chemical to the feces.

The companion animal management apparatus may further include a first sensor unit mounted onto the main body and configured to measure a distance to a companion animal, and a controller configured to control the driving module to maintain the distance to the companion animal within a preset range based on information measured by the first sensor unit.

The companion animal management apparatus may further include a distribution module configured to distribute food to the outside of the main body, and a controller configured to calculate activity of the companion animal based on data about a movement amount of the companion animal management apparatus and operate the distribution module based on data about the calculated activity.

The companion animal management apparatus may further include a controller configured to machine-learn and store a travel route of the companion animal management apparatus and store a contaminated location on the travel route, at which the cleaning module has been operated. The controller may first move the companion animal management apparatus to the contaminated location by operating the driving module.

### MODE FOR INVENTION

As the present disclosure allows for various changes and numerous embodiments, exemplary embodiments will be illustrated in the drawings and described in detail in the written description. The effects and features of the present disclosure as well as methods for achieving the same may be clarified by referring to the following detailed embodiments along with the drawings. The present disclosure may, however, be embodied in many different forms and should not be construed as limited to the exemplary embodiments set forth herein.

Hereinafter, embodiments of the present disclosure are explained in detail referring to the attached drawings. When referring to the drawings, like reference numerals may denote like or corresponding components, and any redundant descriptions thereof are omitted.

As used herein, the singular forms “a,” “an” and “the” are intended to include the plural forms as well, unless the context clearly indicates otherwise.

It will be further understood that the terms “comprises” and/or “includes” used herein specify the presence of stated features or components, but do not preclude the presence or addition of one or more other features or components.

When a certain embodiment may be implemented differently, a specific process order may be performed differently from the described order. For example, two consecutively described processes may be performed substantially at the same time or performed in an order opposite to the described order.

Sizes of components in the drawings may be exaggerated for convenience of explanation. In other words, since sizes and thicknesses of components in the drawings are arbitrarily illustrated for convenience of explanation, the present disclosure is not limited thereto.

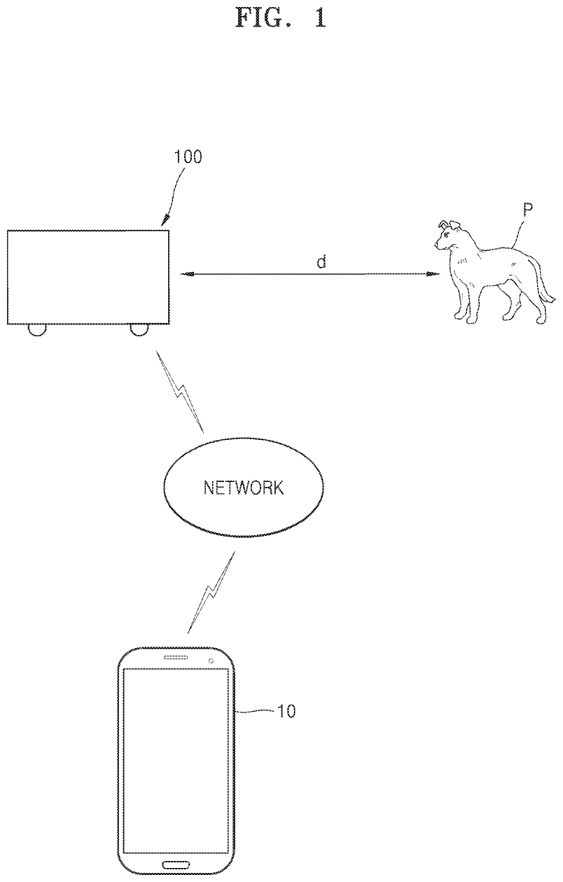

FIG. 1 is a diagram of a companion animal management system 1 according to an embodiment of the present disclosure.

With reference to FIG. 1, according to the companion animal management system 1, a companion animal management apparatus 100 may move about in a space in which a companion animal P is present and obtain information about the companion animal P. The companion animal management system 1 may control the companion animal management apparatus 100 through a user terminal 10.

The companion animal management apparatus 100 may move along with the companion animal P. The companion animal management apparatus 100 may monitor the companion animal P in real time and obtain activity and bio-data of the companion animal P.

According to the companion animal management apparatus 100, a user may remotely control the companion animal management apparatus 100 through the user terminal 10. In addition, the user terminal 10 may receive information about the companion animal P from the companion animal management apparatus 100.

The user terminal 10 may refer to a communication terminal which may be used in a wired or wireless communication environment. For example, the user terminal 10 may be a smartphone, a tablet personal computer (PC), a PC, a smart TV, a personal digital assistant (PDA), a laptop, a media player, a micro server, a global positioning system (GPS) device, an electronic book reader, a digital broadcasting terminal, a navigation, a kiosk, a MP3 player, a digital camera, a home appliance, a device with a camera, and other mobile or non-mobile computing devices. In addition, the user terminal 10 may be a wearable device, such as a watch, glasses, hair band, and ring which have communication function and data processing function. However, any terminal with an application enabling the internet communication as described above may be adopted without limitation.

The user terminal 10 and the companion animal management apparatus 100 may perform communication by using a network. For example, the network may include a local area network (LAN), a wide area network (WAN), a value added network (VAN), a mobile radio communication network, a satellite network, or a combination thereof, and may refer to a data communication network in comprehensive sense which facilitates communication among constituents of the network and includes wired internet, wireless internet, and mobile wireless communication network. In addition, the wireless communication may include wireless fidelity (Wi-Fi), Bluetooth, Bluetooth low energy, ZigBee, Wi-Fi direct (WFD), ultra wideband (UWB), infrared data association (IrDA), near field communication (NFC), 5G, etc.; however, the present disclosure is not limited thereto.

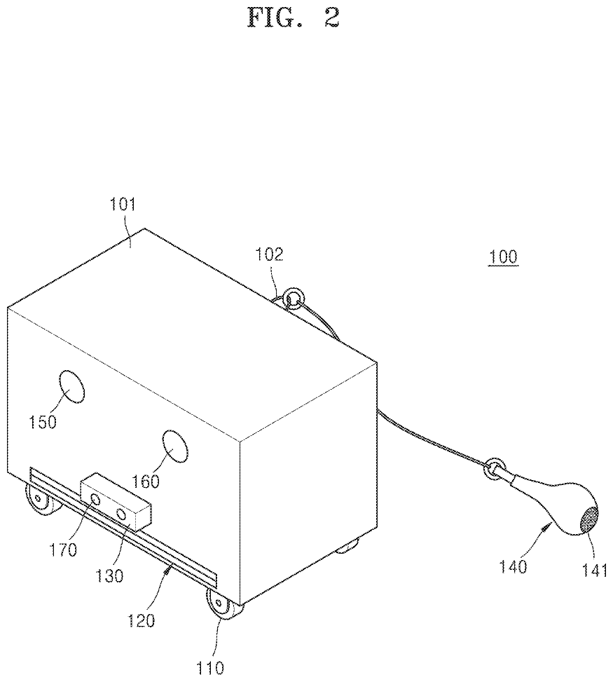

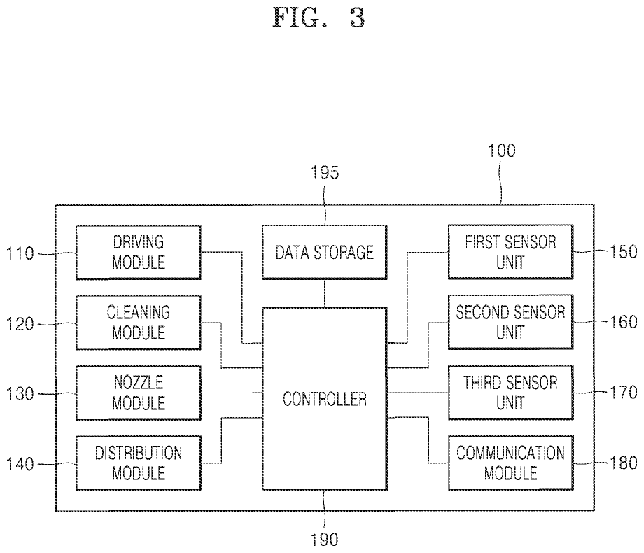

FIG. 2 is a diagram of the companion animal management apparatus 100 according to an embodiment of the present disclosure, and FIG. 3 is a block diagram illustrating some components of the companion animal management apparatus 100 of FIG. 2.

With reference to FIGS. 2 and 3, the companion animal management apparatus 100 may include a driving module 110, a cleaning module 120, a nozzle module 130, a distribution module 140, a first sensor unit 150, a second sensor unit 160, a third sensor unit 170, a communication module 180, and a controller 190. In addition, a data storage 195 connected to the controller 190 may further provided to store data measured by the first sensor unit 150, the second sensor unit 160, and the third sensor unit 170.

A main body 101 may form the exterior of the companion animal management apparatus 100. Each component of the companion animal management apparatus 100 may be built in an interior space of the main body 101. Although the drawings illustrate that the main body 101 has a cuboid shape; however, the present disclosure is not limited thereto, and the main body 101 may have various shapes.

The driving module 110 may move the main body 101. For example, the driving module 110 may be mounted at the bottom of the main body 101 and drive wheels with driving power generated from a motor (not shown) and a battery (not shown).

The cleaning module 120 may be arranged in the interior space of the main body 101. The cleaning module 120 may remove faces of the companion animal P. The cleaning module 120 may process feces inside the main body 101 by protruding out from the front of the main body 101 and retreating back into the main body 101.

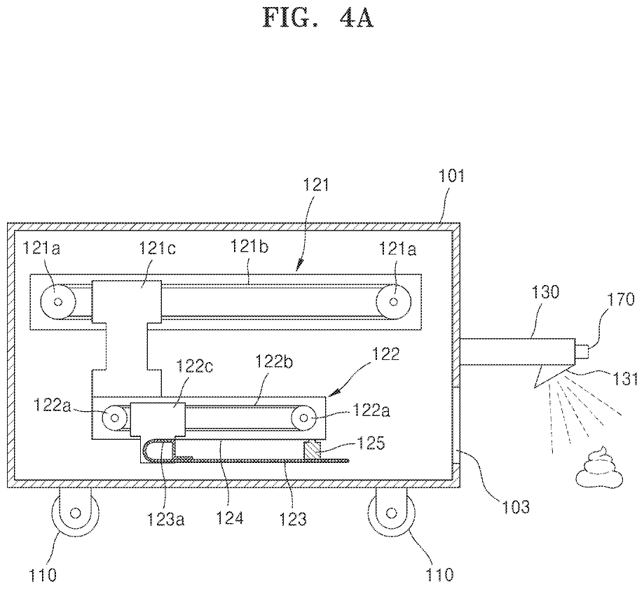

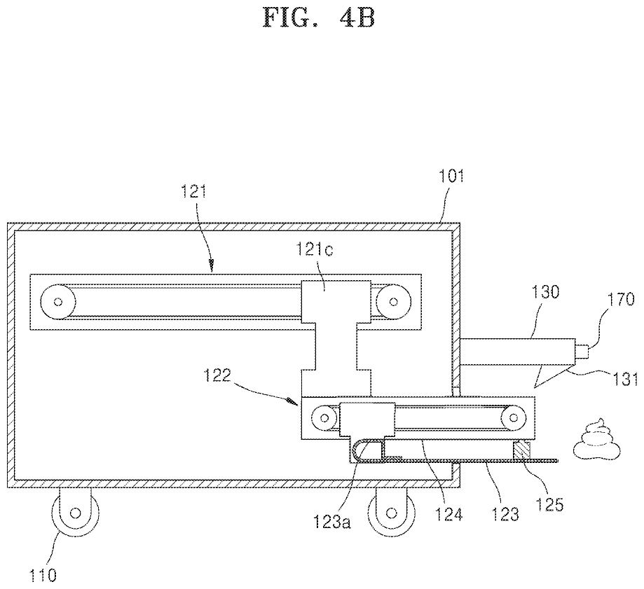

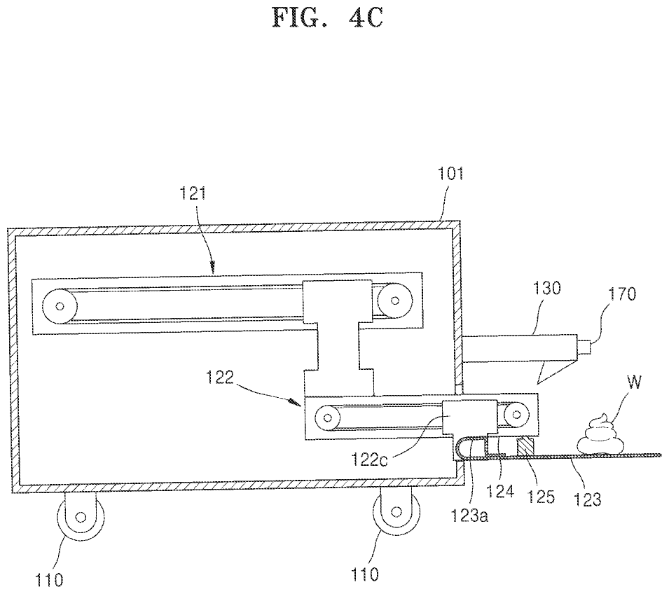

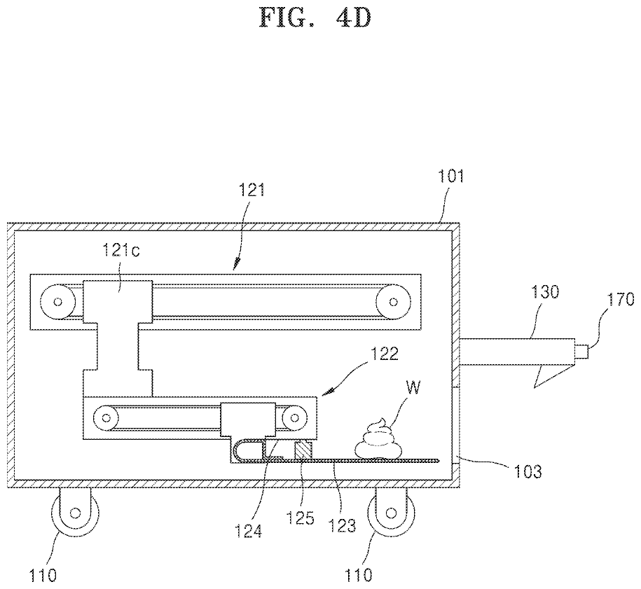

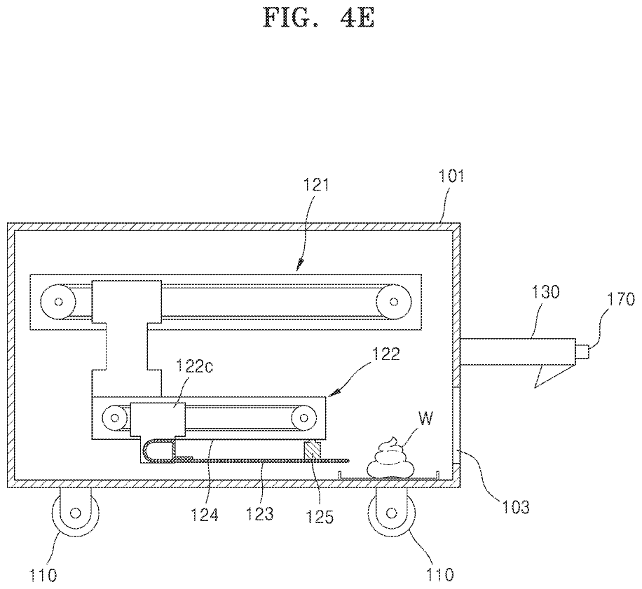

With reference to FIG. 4A, the cleaning module 120 may include a first moving unit 121, a second moving unit 122, a plate 123, a cover film 124, and a connection block 125. The first moving unit 121 may be installed in the main body 101. The second moving unit 122 may be connected to the first moving unit 121 and linearly move according to operation of the first moving unit 121.

The first moving unit 121 may be installed in the interior space of the main body 101, and may include a first driving portion 121a, a first connecting portion 121b, and a first moving portion 121c.

The first driving portion 121a may include a pair of driving wheels, and the first connecting portion 121b may connect the first driving portions 121a. The first connecting portion 121b may include various components, such as a chain, a belt wire, etc.

The first moving portion 121c may linearly move according to operation of the first driving portion 121a. The first moving portion 121c may be mounted onto the first connecting portion 121b and linearly reciprocate between the first driving portions 121a.

The second moving unit 122 may be connected to the first moving portion 121c and linearly move along with the first moving unit 121. The second moving unit 122 may include a second driving portion 122a, a second connecting portion 122b, and a second moving portion 122c.

The second moving unit 122 may be connected to the first moving portion 121c and may be linearly moved by the operation of the first moving portion 121c. In addition, the second moving portion 122c may be linearly moved by the second driving portion 122a.

The second driving portion 122a may include a pair of driving wheels, and the second connecting portion 122b may connect the second driving portions 122a. The second connecting portion 122b may include various components, such as a chain, a belt wire, etc.

The second moving portion 122c may linearly move according to operation of the second driving portion 122a. The second moving portion 122c may be mounted onto the second connecting portion 122b and linearly reciprocate between the second driving portions 122a.

The plate 123 may be connected to the second moving portion 122c. The plate 123 may be linearly reciprocated according to the linear movement of the second moving portion 122c.

The plate 123 may extend in certain length and may include a first end 123a and a second end 123b. The cover film 124 may be turned at the first end 123a, having a certain height, and the cover film 124 may be arranged on the upper and lower surfaces of the second end 123b. An area covering the upper surface of the second end 123b may vary according to the movement of the second moving portion 122c.

The first end 123a of the plate 123 may be mounted onto the second moving portion 122c. The first end 123a may be bent and an outer surface of the first end 123a may have a curvature. The first end 123a may have a certain height and the cover film 124 may be turned. An upper portion and a lower portion of the first end 123a may have a flat shape, and a lateral side of the first end 123a may have a curved surface.

The second end 123b of the plate 123 may be a pointed at the end. The cover film 124 may extend on a lower surface of the second end 123b, and partially cover an upper surface of the second end 123b. More specifically, the cover film 124 may cover from the end point of the second end 123b to the connection block 125.

A part of the cover film 124 may cover the plate 123. Both ends of the cover film 124 may be fixed to the connection block 125 arranged outside the second moving unit 122, and an area covering the upper surface of the plate 123 may be changed according to linear movement of the second moving portion 122c.

One end of the cover film 124 may be fixed to the connection block 125 and extend to the upper surface of the first end 123a of the plate 123. The cover film 124 may be turned at the first end 123a and cover the lower surface of the plate 123. The cover film 124 may be turned at the second end 123b, cover the upper surface of the second end 123b, extend to the connection block 125, and be fixed to the connection block 125.

The connection block 125 may be mounted onto the second moving unit 122. The connection block 125 may be mounted onto the front portion of the second moving unit 122. A second connection block 125 may fix the cover film 124. As both ends of the cover film 124 are fixed to the second connection block 125, the cover film 124 may cover the plate 123, and circulate along with the plate 123.

The nozzle module 130 may be arranged on the front of the main body 101 and spray liquid chemical. The nozzle module 130 may store the liquid chemical therein, and the liquid chemical may be sprayed through a nozzle 131.

The nozzle module 130 may spray liquid chemical to process feces. For example, the nozzle module 130 may spray a liquid chemical hardening the feces, a liquid chemical minimizing odor, etc.

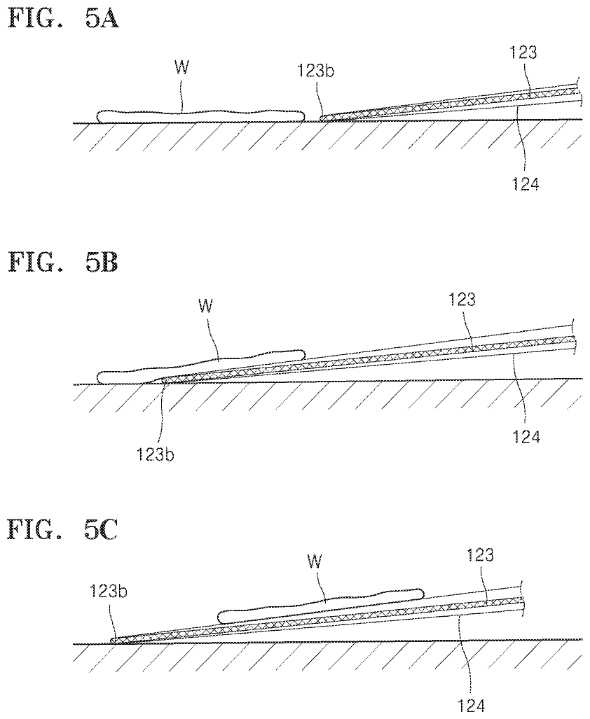

FIGS. 4A to 4E are each a cross-sectional diagram illustrating operation of the companion animal management apparatus 100 of FIG. 2, and FIGS. 5A-5C are cross-sectional diagrams illustrating operation of the cleaning module 120 of FIG. 3.

With reference to FIGS. 4A to 5C, a method of removing feces by the cleaning module 120 is described.

FIG. 4 illustrates that, when feces are detected by at least one of the first sensor unit 150 to third sensor unit 170, the cleaning module 120 may prepare to operate. When the feces need to be preprocessed, the nozzle module 130 may spray liquid chemical thereon. The nozzle module 130 may spray the liquid chemical frontward to preprocess the feces.

With reference to FIG. 4B, the cleaning module 120 may operate the first moving unit 121. When the first moving unit 121 is operated, the first moving portion 121c may move frontward, moving the second moving unit 122 frontward as well.

With reference to FIG. 4C, the feces W may be loaded on the cleaning module 120. When the cleaning module 120 operates the second moving unit 122, the second moving portion 122c may move frontward. When the second moving portion 122c moves frontward, the plate 123 may also move frontward, and the feces W may be loaded on the upper surface of the plate 123.

More specifically, in FIGS. 5A-5C, as the plate 123 moves frontward, the second end 123b goes beneath the feces W. In this regard, the cover film 124 may also move along with the plate 123, and the feces W may be placed on the cover film 124. When the plate 123 moves forward, the cover film 124 may move the feces W backwards.

With reference to FIG. 4D, the first moving unit 121 may be operated to move the first moving portion 121c backwards. When the first moving portion 121c moves backwards, the second moving unit 122 may also move backwards. Then, the feces W placed on the plate 123 may be moved into the main body 101.

With reference to FIG. 4E, the second moving unit 122 may be operated to move the second moving portion 122c backwards. When the second moving portion 122c moves backwards, the plate 123 may also move backwards. Accordingly, the feces W placed on the cover film 124 may be separated from the cover film 124 and fall on a processing portion 126.

Referring back to FIGS. 2 and 3, the distribution module 140 may be provided on one side of the main body 101 to distribute treats, etc. to the companion animal P.

For example, the distribution module 140 may be coupled to a connection band 102 of the main body 101. The distribution module 140 may store treats, etc. therein, and discharge treats, etc. through an opening 141.

Alternatively, the distribution module 140 may be mounted inside the main body 101 and be opened at a set time to distribute treats to the companion animal P.

The first sensor unit 150 may be mounted onto one side of the main body 101 and obtain an image. A camera, etc. may be provided as the first sensor unit 150. The first sensor unit 150 may sense an object, feces, etc. that lie in front thereof.

The second sensor unit 160 may be mounted onto one side of the main body 101 and sense presence of objects. For example, the second sensor unit 160 may be an ultrasonic sensor or an infrared sensor. The second sensor unit 160 may sense whether there is an object in front thereof.

The third sensor unit 170 may be mounted onto one side of the main body 101 and sense odor of the feces W. The third sensor unit 170 may be defined as a sensor configured to measure odors, and may measure odors generated from the feces W. Based on data measured by the third sensor unit 170, the nozzle module 130 may spray liquid chemical.

The communication module 180 may be connected to the user terminal 10 and receive data from the outside or receive control signals.

The controller 190 may control operations of each component of the companion animal management apparatus 100. The controller 190 may be connected to the driving module 110 and move the companion animal management apparatus 100. The controller 190 may operate the cleaning module 120 based on data measured by the first to third sensor units 150, 160, and 170. In addition, the controller 190 may control open/close of the distribution module 140.

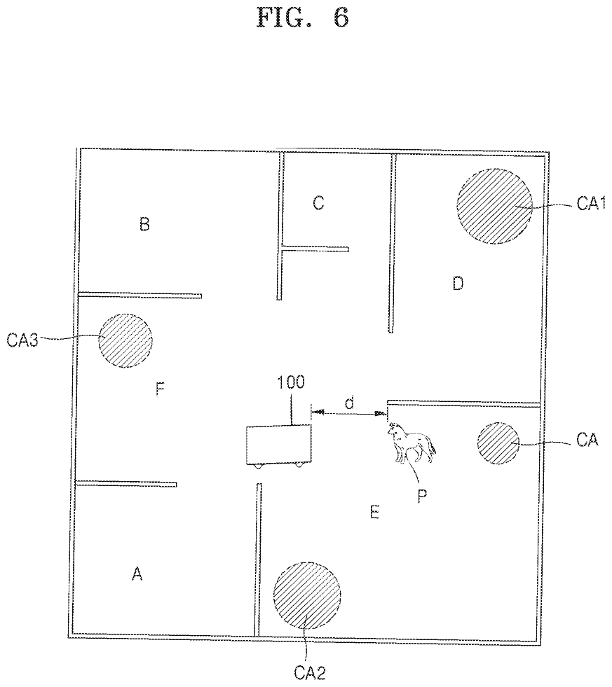

FIG. 6 is a diagram illustrating a method of controlling operation of the companion animal management apparatus 100 of FIG. 2.

With reference to FIG. 6, the companion animal management apparatus 100 may map a management target space and set a cleaning route based on the history of cleaning.

<Cleaning Mode>

In the cleaning mode, the companion animal management apparatus 100 may estimate an expected location of defecation by using stored previous data. In addition, the companion animal management apparatus 100 may select a travel route based on the expected location of defecation.

The controller 190 may machine-learn and store the travel route of the companion animal management apparatus 100, and store a contaminated location on the travel route, at which the cleaning module 120 has operated. When the cleaning module 120 has cleaned feces in a first contaminated area CA1, a second contaminated area CA2, a third contaminated area CA3, and a fourth contaminated area CA4, the controller 190 may store a travel route and contaminants accordingly in the data storage 195. The controller 190 may later set a travel route including all of the first to fourth contaminated areas CA1 to CA4.

In addition, the controller 190 may operate the driving module 110 to first move the companion animal management apparatus to a contaminated location. For example, when the companion animal management apparatus 100 is in the cleaning mode, the driving module 110 may be operated such that the companion animal management apparatus 100 may first move to the first contaminated area CA1, the second contaminated area CA2, the third contaminated area CA3, and the fourth contaminated area CA4.

The controller 190 may set a space managed by the companion animal management apparatus 100 based on image data measured by the first sensor unit 150 or object data measured by the second sensor unit 160. For example, the controller 190 may distinguish and map sections A to F and objects inside the house.

The companion animal management apparatus 100 may set a travel route based on an area in which the feces W have been cleaned previously.

For example, when the feces have been cleaned previously in the first contaminated area CA1, the second contaminated area CA2, the third contaminated area CA3, and the fourth contaminated area CA4, the companion animal management apparatus 100 may set a route to first clean the sections E, F, and D.

As the companion animals P defecate at a particular spot, the companion animal management apparatus 100 may clean in priority the locations in which the companion animal P is expected to defecate based on previous cleaning history data to quickly maintain the clean environment.

In another example, the controller 190 may use data about contaminated areas to set an order of priority of sections to be cleaned. For example, a range of contaminated areas in each section may be set based on the cleaning history stored in the data storage 195, and a section to be cleaned first may be selected based on the set range.

For example, when it is identified that the section D includes the first contaminated area CA1, the section E includes the second contaminated area CA2 and the fourth contaminated area CA4, and the section F includes the third contaminated area CA3, the section E may be selected as a section to be cleaned first. Moreover, by calculating a ratio of the third contaminated area CA3 in the entire area of the section F, and a ratio of the first contaminated area CA1 per area of the section D, a section having a higher ratio may be selected as a section to be cleaned next.

The companion animal management apparatus 100 may first clean the sections which have been cleaned previously and then clean sections which have never been cleaned. For example, after the sections D, E, and F are cleaned, the sections A, B, and C may be cleaned.

<Activity Estimation Mode>

The companion animal management apparatus 100 may measure information or condition of the feces W of the companion animal P. For example, the controller 190 may measure an amount and a weight of the feces W collected by the cleaning module 120 and compare the same with previous data. Based on this, condition of the companion animal P may be estimated. For example, when the amount of the feces W decreases rapidly, or the condition of the feces W is not normal, the user terminal 10 may receive information about such condition.

In addition, the activity of the companion animal P may be measured, and the measured activity may be compared with the data pre-stored in the data storage 195 to estimate the health condition of the companion animal P. Moreover, based on the activity data, the controller 190 may adjust an amount of food discharged by the distribution module 140.

<Activity Enhancement Mode>

The companion animal management apparatus 100 may observe locations of the companion animal P and enhance activity of the companion animal P. The companion animal management apparatus 100 may identify a location of the companion animal P by using the first sensor unit 150 or the second sensor unit 160.

For example, when the companion animal management apparatus 100 is set to maintain a certain distance d from the companion animal P, the companion animal management apparatus 100 may move along with the companion animal P. That is, the companion animal management apparatus 100 may intrigue the companion animal P to enhance activity of the companion animal P.

The companion animal management system 1 and the companion animal management apparatus 100 according to the present disclosure may monitor condition of the companion animal P and deliver the same to the user terminal 10 such that the user may check and manage the condition of the companion animal in real time. The companion animal management apparatus 100 may obtain a current image of the companion animal P such that the user may check on the companion animal in real time.

According to the companion animal management system 1 and the companion animal management apparatus 100 of the present disclosure, the companion animal management apparatus 100 may obtain data about activity of the companion animal P such that condition of the companion animal P is closely managed. By measuring data about feces, movement distance, etc. of the companion animal P, condition of the companion animal P may be assumed, and an amount of food or treat to be fed may be adjusted.

The companion animal management system 1 and the companion animal management apparatus 100 according to the present disclosure may clean up feces of the companion animal P. The cleaning module 120 may accurately collect and remove the feces W. In addition, the nozzle module 130 may spray liquid chemical to remove odor of feces and quickly harden the feces.

The companion animal management system 1 and the companion animal management apparatus 100 according to the present disclosure may set a cleaning route based on data about a position where the companion animal P has lastly defecated. By first cleaning the position where the companion animal P has defecated, dispersion of contamination may be prevented.

The technical ideas of the present disclosure are not limited to the embodiments described above, and it is to be appreciated that all changes, equivalents, and substitutes that do not depart from the spirit and technical scope of the claims are encompassed in the scope of the technical ideas of the present disclosure.

1. A companion animal management apparatus comprising:
a main body;
a driving module configured to move the main body; and
a cleaning module installed in an interior space of the main body and including a part which protrudes out to a front of the main body and retreats back into the main body to process feces inside the main body.

2. The companion animal management apparatus of claim 1, wherein the cleaning module comprises:
a first moving unit fixed to the main body and configured to linearly move a first moving portion;
a second moving unit connected to the first moving portion, linearly moving along with the first moving unit, and configured to linearly move a second moving portion;
a plate connected to the second moving portion; and
a cover film including both ends fixed to a connection block arranged outside the second moving unit and having an area covering an upper surface of the plate, the area changing according to linear movement of the second moving portion.

3. The companion animal management apparatus of claim 1, further comprising a spray module arranged on the front of the main body and configured to spray liquid chemical to the feces.

4. The companion animal management apparatus of claim 1, further comprising:
a first sensor unit mounted onto the main body and configured to measure a distance to a companion animal; and
a controller configured to control the driving module to maintain the distance to the companion animal within a preset range based on information measured by the first sensor unit.

5. The companion animal management apparatus of claim 1, further comprising:
a distribution module configured to distribute food to an outside of the main body; and
a controller configured to calculate activity of the companion animal based on data about a movement amount of the companion animal management apparatus and operate the distribution module based on data about the calculated activity.

6. The companion animal management apparatus of claim 1, further comprising a controller configured to machine-learn and store a travel route of the companion animal management apparatus and store a contaminated location on the travel route, at which the cleaning module has been operated.

7. The companion animal management apparatus of claim 6, wherein the controller is further configured to first move the companion animal management apparatus to the contaminated location by operating the driving module.
